(19) United States
(12) Patent Application Publication
(10) Pub. No.: US 2004/0072237 A1
(43) Pub. Date: Apr. 15, 2004

(54) Use of cytokines secreted by dendritic cells

(76) Inventor: Barry Schweitzer, Woodbridge, CT (US)

Correspondence Address: BANNER & WITCOFF, 1001 G STREET N W, SUITE 1100, WASHINGTON, DC, 20001, US

(21) Appl. No.: 10/291,797

(22) Filed: Nov. 12, 2002

Related U.S. Application Data
(60) Provisional application No. 60/342,093, filed on Dec. 26, 2001.

(51) Int. Cl. G01N 33/53; G01N 33/567

(52) U.S. Cl. 435/7.1; 435/7.2

(57) ABSTRACT

A prerequisite of proteomics is the ability to quantify many selected proteins simultaneously. Fluorescent sandwich immunoassays on microarrays hold appeal for such studies, since equipment and antibodies are readily available, and assays are simple, scalable and reproducible. To attain adequate sensitivity and specificity, however, a general method of immunoassay amplification is required. Coupling of isothermal rolling circle amplification (RCA) to universal antibodies can be used for this purpose: RCA on a synthetic DNA circle is initiated by a complementary oligonucleotide attached to an anti-biotin antibody; single-stranded RCA product remains attached to the antibody, and is detected by hybridization of complementary, fluorescent oligonucleotides. 51 cytokines were measured simultaneously on microarrays with signal amplification by RCA with high specificity, femtomolar sensitivity and 4 log quantitative range. This cytokine microarray was used to measure secretion from human Dendritic cells (DCs) induced by lipopolysaccharide (LPS) or tumor necrosis factor-alpha (TNF-(α). Rapid secretion of inflammatory cytokines such as MIP-1β, IL-8, and IP-10 was induced by LPS. Eotaxin-2 and I-309 were found to be induced by LPS, and MDC, TARC, sIL-6R, and sTNF-RI were found to be induced by TNF-α. Since microarrays can accommodate ˜1000 sandwich immunoassays of this type, a relatively small number of RCA microarrays appears to offer a tractable approach for proteomic surveys.

### FIELD OF THE INVENTION

The invention relates to the field of immunology. In particular it relates to the cytokines released by dendritic cells which influence the differentiation pathways of naive T cells.

### BACKGROUND OF THE PRIOR ART

Ordered arrays of proteins provide an attractive strategy for high-throughput analysis of proteins. To be truly useful for this purpose, however, such arrays must yield sensitive, quantitative, and reproducible measurements of protein levels. It is also desirable that assays on these arrays utilize small sample volumes and be amenable to automated systems for high-throughput processing. There have been a number of recent examples of the use of protein arrays for a variety of applications (1-6). While these approaches have established the feasibility of protein arrays, they have not yet demonstrated practical utility for measuring protein expression levels in a manner analogous to a gene expression array. A microarray consisting of immobilized antibodies is the most straightforward near-tern approach for developing a chip for highly parallel analysis of protein levels. Experience with such arrays is limited, and the levels of sensitivity (ca. 10 ng/mL) and multiplexing have been insufficient for quantifying most biological change (7-10).

In the present study, we establish the utility of RCA (11-13) for high-throughput analysis of protein expression on microarrays, providing assays that are highly sensitive, quantitative, and reproducible. We describe highly-multiplexed, microarray immunoassays with 4 steps: sample application and protein capture by specific antibodies affixed to a microarray, binding of second antibodies to captured proteins, binding of an universal antibody to the second antibodies, and RCA signal amplification on the universal antibody. This approach was used to examine the time course of cytokine secretion by human Dendritic cells (DCs) cultured with or without the maturation agents LPS (lipopolysaccharide) or TNF-α (tumor necrosis factor-α; 14). There is a need in the art for additional definition of the components and interactions of the components of the immune system, so that they can be manipulated for diagnostic and therapeutic benefit.

### BRIEF SUMMARY OF THE INVENTION

In a first embodiment a method is provided of diagnosing an inflammatory syndrome in a patient. Altered expression of one or more cytokines selected from the group consisting of: IL-6, IL-11, IL-1β, VEGF, ANG, I-309, sTNF-R1, Eot-2, and sIL-6R is determined in a test sample from the patient. The test sample can be, for example, serum, plasma, blood, lymph fluid, peripheral lymphatic tissue, or blood. Determination of altered expression of one or more of the cytokines is used to aid in forming a diagnosis of an inflammatory syndrome.

In a second embodiment of the invention a method of treating a patient with an inflammatory syndrome is provided. One or more antibodies which specifically bind to a cytokine selected from the group consisting of: IL-6, IL-11, IL-1β, VEGF, ANG, I-309, sTNF-R1, Eot-2, and sIL-6R are administered to a patient with an inflammatory syndrome. The amount of one or more of the cytokines is consequently reduced in the patient.

The invention thus provides the art with diagnostic and therapeutic methods for clinically managing inflammatory syndromes.

### BRIEF DESCRIPTION OF THE DRAWINGS

FIG. 1. Cartoon of multiplexed immunoassays on microarrays with RCA signal amplification. 150 spots are printed with a microarray device onto each of 16 separate analysis areas (“subarrays”) on a Teflon-coated glass slide. The identity and location of each spot is known. Immunoassays are performed on these protein microarrays as follows: In step 1, a 10 μL sample is applied to the analysis area for 30 min, and then washed off. The printed antibodies capture specific proteins of interest from the sample. Captured proteins are recognized in step 2 by applying a mixture of biotinylated secondary antibodies that are matched with each printed capture antibody. In step 3, detection of secondary antibodies is carried out using an anti-biotin antibody to which has been attached a primer that is hybridized to a DNA circle. RCA makes copies of this DNA circle, starting at the primer attached to the anti-biotin antibody, and continuing to generate a single, long strand of DNA that is composed of thousands of copies of the circle sequence. RCA product is detected by specific, complementary, labeled DNA probes. RCA product fluorescence is measured with a conventional microarray scanning device. The amount of fluorescence at each spot is directly proportional to the amount of specific protein in the original sample.

FIG. 2. Sensitivity of single cytokine detection by RCA and direct detection.

Serial dilutions of individual cytokines were incubated on duplicate subarrays containing 25 different printed antibodies, each printed 4 times. After incubation and washing, a biotinylated polyclonal antibody specific to MIP-1β, IL-1α, or IL-8 was added to the corresponding subarrays. On one set of subarrays, detection was carried out using RCA signal amplification. On the second set of subarrays, “direct” detection was performed using Cy5-labeled streptavidin. Fluorescence intensity of each spot was measured with a microarray scanner, and averages of the 8 replicates of each antibody were plotted.

FIG. 3. Specificity of single cytokine detection on protein microarrays by RCA.

Solutions of MCP-1 or FGF-7 (1 ng/ml) were incubated on separate pre-blocked subarrays on which had been printed duplicate spots representing each of 24 different anti-cytokine antibodies. Following incubation and washing, a mixture of 24 biotinylated polyclonal detector antibodies was added to each microarray. After incubation and washing, RCA was carried out, fluorescence intensity of each spot was measured with a microarray scanner, and average values for each cytokine were plotted. FIG. 3A. Quantitation of fluorescence of the subarray incubated with MCP-1. FIG. 3B. Quantitation of fluorescence of the subarray incubated with FGF-7.

FIG. 4. Sensitivity and specificity of multiplex detection on protein microarrays by RCA.

FIG. 4A. 7 cytokines were mixed, serially diluted in PBS (from 600 pg/mL to 0.1 pg/mL), and incubated on pre-blocked subarrays containing monoclonal antibodies spotted in quadruplicate columns. After incubation and washing, a mixture of the corresponding biotinylated polyclonal detector antibodies were added to each subarray, and RCA was performed. Shown are fluorescence images of subarrays obtained with a microarray scanner. Top row of quadruplicate columns, left to right: MIP-1β, TARC, MCP-1, RANTES; Bottom row of quadruplicate columns, left to right: sIL-6R, MDC, I-309, Biotin-mIgG (positive control).

FIG. 4B. Quantitation of fluorescence from images shown in A. Indicated on each histogram are mean fluorescence intensities and standard deviations derived from 2 subarrays, with 4 spots per subarray.

FIG. 4C. A mixture of IL-6, IL-8, IL-2, MCP-1, IL-18, EGF, TNF-α, IL-1β, NGF-β, IL-1α, and ENA-78 at 100 pg/mL in PBS/Tween (Bottom Panel) or PBS/Tween (Top Panel) was added to a pre-blocked subarray. After incubation and washing, a mixture of biotinylated polyclonal detector antibodies for IL-2, IL-6, IL-8, and MCP-1 was added to each subarray, RCA was performed and fluorescence was measured with a microarray scanner. Fluorescent spots at the bottom of both images represent Biotin-mIgG (positive control).

FIG. 5. Kinetics of cytokine production in maturing LCs on microarrays.

Cytokine levels present in LC culture supernatants at 6 time points without induction or after LPS or TNF-α stimulation were determined by microarray immunoassay. Fluorescence intensities were converted to pg/mL using standard curves generated from mixtures of purified cytokines serially diluted in X-VIVO culture medium. The data for IL-8, MDC, TARC, and sIL-6R were generated from experiments using 1:20 dilutions of culture supernatants, and were corrected for this dilution factor. Black circles—LPS-treated; red triangles—TNF-α; green squares—uninduced.

FIG. 6. Cytokines with at least 4-fold increased abundance during 72 hour culture with or without LPS or TNF-α.

FIG. 7. Comparison of MDC measurement in supernatants by commercial ELISA and multiplexed RCA-amplified microarray immunoassay.

MDC levels present in LC culture supernatants at 6 time points without induction or after LPS or TNF-α stimulation were determined by RCA microarray immunoassay (above) or commercial ELISA (below). Fluorescence intensities of the RCA microarray immunoassay were converted to pg/mL using standard curves generated from mixtures of purified cytokines serially diluted in X-VIVO culture medium. 10 μl of 1:20 dilutions of culture supernatants were used for RCA microarray immunoassay. 200 μl of neat culture supernatants were assayed for MDC using a 96-well ELISA kit (R&D Systems, Minneapolis, Minn.) according to the manufacturer's instructions. Black circles—LPS-treated; red triangles—TNF-α; green squares—uninduced.

### DETAILED DESCRIPTION OF THE INVENTION

It is a discovery of the present inventors that certain cytokines are secreted by human dendritic cells (Langerhans cells, in particular) when they are induced to differentiate with lipopolysaccharide or tumor necrosis factor-α. Lipopolysaccharide induces Langerhans cells to differentiate and secrete cytokines which stimulate naive T helper cells to differentiate to T_{H1} helper cells which stimulate cellular immune responses. Tumor necrosis factor-α induces Langerhans cells to differentiate and secrete cytokines which stimulate naive T helper cells to differentiate toward T_{H2} helper cells which stimulate humoral immune responses. Identification of these cytokines permits their use diagnostically and therapeutically in relation to inflammatory syndromes.

Three cytokines (TARC, MDC, MCP-1) were found that were secreted by uninduced cells as well as by cells induced by LPS or TNF-α. Seven cytokines (I-309, MIP-1β, IP-10, RANTES, sTNF-RI, Eot-2, sIL-6R) were identified that were secreted upon induction by TNF-α and upon induction by LPS. One cytokine (ANG) was only expressed upon induction with TNF-α. Six cytokines (IL-6, IL-8, IL-11, IL-12, IL-1β, and VEGF) were expressed only upon induction with LPS. The latter two classes are likely to be critical in determining whether naive helper T cells become committed to the cellular or humoral immunity pathway. The identified cytokines secreted by induced Langerhans cells are useful in diagnosing inflammatory syndromes. Antibodies which specifically bind to the cytokines can be used to therapeutically treat inflammatory syndromes.

Inflammatory syndromes which can be advantageously diagnosed and treated according to the present invention include sepsis, arthritis, allergy, enteritis, severe acute pancreatitis, emphysema, multiple organ failure, and acute respiratory distress syndrome (ARDS). Other inflammatory syndromes are also amenable to the methods of the invention.

Test samples used for performing the diagnostic method are preferably from serum, plasma, blood, lymph fluid, peripheral lymphatic tissue, or blood. Desirably the test sample contains, or has contained, dendritic cells, and more preferably Langerhans cells. However, it may be desirable that the actual sample upon which the assay is performed be relatively free of cells.

Altered expression of a cytokine can be determined relative to a control sample. The control sample can be obtained from an organ distal to the area of local inflammation in the test subject. Alternatively the control sample can be obtained from a subject not experiencing or evidencing an inflammatory syndrome. Altered expression can be determined at any threshold which is statistically significant. This can be an increase relative to a control sample of 25%, 50%, or 75%, for example. The threshold can be set to at least two-fold the level of the control sample. Alternatively, the threshold can be set to at least three-fold the level in the control sample. A more stringent threshold can be set to at least four-fold the level in the control sample.

Altered expression of a cytokine can be determined using either mRNA or protein as an indication of expression level. Preferably the protein will be determined. The determination need not be strictly quantitative. For example, in cases where a cytokine goes from an unexpressed to an expressed state a qualitative assessment may be sufficient. Any assay known in the art for detecting gene expression can be used, either individually or multiplexed. The assays used may involve gene arrays, protein arrays, antibody arrays, Western blotting, ELISA, immunoprecipitation, filter binding assays, hybridization assays, etc. The protein microarray employing a rolling circle amplification for detection described in detail below is preferred, but need not be used. Briefly, capture antibodies are affixed to a solid support in a predetermined pattern (array) and test sample is applied to the array so that proteins (cytokines) in the test sample can bind to antibodies on the array which are specific for that particular protein. Second antibodies are applied which are specific for the same set of proteins as are the capture antibodies. The second set of antibodies can be labeled with a hapten. A third set of antibodies is then applied to the array. The third set of antibodies is specific for the hapten on the second set of antibodies or with the constant region of the second set of antibodies. The third set of antibodies contains an attached oligonucleotide. The oligonucleotide can be used as a primer to amplify a template to create an amplification signal. Preferably the template is a circular DNA such that rolling circle amplification can create a large signal. Alternatively, the second antibody can be directly detectable, for example by rolling circle amplification of an attached oligonucleotide.

Unwanted immune reactions associated with inflammatory syndromes can be treated by administering an antibody which specifically binds to one of the cytokines identified here as secreted by stimulated, maturing Langerhans cells. The antibody can be a monoclonal or polyclonal antibody. It can be a complete antibody molecule or a fragment. Standard antibody fragments are known in the art and any of these can be used, including Fab, F(ab′)₂ Single chain Fv (ScFv) can also be used. The antibodies can if desired be attached to other moieties, such as therapeutic agents. Single antibodies or cocktails of antibodies can be used. The cocktails can be directed to the same or different cytokines. Antibodies can be administered by any means known in the art, including but not limited to intravenous, intrathecal, directly to the thymus or to a lymph nodes, subcutaneous, oral, and intramuscular.

### EXAMPLES

### Example 1

### Experimental Protocol

Conjugate Synthesis. Mouse monoclonal anti-biotin IgG (Jackson ImmunoResearch Laboratories, Inc.) at a concentration of 5 mg/ml in PBS (phosphate buffered saline) buffer (pH 7.2) with 10 mM EDTA was reduced with 2-mercaptoethylamine (MEA, Pierce Chemical Co.) for 90 min at 37° C. The reduced IgG was purified on a PD10 column (Amersham Pharmacia Biotech, Piscataway, N.J.). A 5′-terminal amine-modified oligonucleotide, 5′-NH₂-AAA AAA AAA AAA AAA CAC AGC TGA GGA TAG GAC AT-3′, was treated with N-[γ-maleimidobutyryloxy]sulfo-succinimide ester (sulfo-GMBS, Pierce Chemical Co.) in PBS buffer (pH 7.2). The reaction was incubated for 30 min at 37° C. and then 30 min at room temperature. The maleimide-activated oligo was purified with a PD10 column. Fractions containing modified oligo were collected and concentrated. The derivatized oligo was then conjugated to the reduced IgG (molar ratio of modified oligo to reduced IgG was 10:1) by incubation for 2 hrs at room temperature. The conjugate was then purified by Superdex 200 gel filtration (Amersham Pharmacia Biotech).

RCA Microarray Immunoassays. Glass slides coated with Teflon except for 16 circular areas or “subarrays” were functionalized with thiol silane and activated with GMBS (12). Monoclonal antibodies (R&D Systems, Minneapolis, Minn.) were diluted to 0.5 mg/ml in PBS with 0.05 mg/ml BSA and spotted onto the slides using a pin-tool type microarrayer (Genemachines, San Carlos, Calif.), and slides were blocked as described (12). A 10 μL volume of sample containing either purified antigen or supernatant from cell cultures was applied to each subarray and incubated for 30 min. After incubation, subarrays were washed twice with 30 μL PBS, 0.5% Brj-35 with a 2 min interval between each wash. A mixture of biotinylated secondary antibodies (25 μL diluted to 0.1 μg/ml in PBS. 0.5% Bij-35) was applied to each subarray, incubated for 30 min and washed as described above.

The anti-biotin antibody conjugate was annealed for 30 min in PBS/0.05%Tween-20/2 mM EDTA at 37° C. with an oligonucleotide (5′-CTC AGC TGT GTA ACA ACA TGA AGA TTG TAG GTC AGA ACT CAC CTG TTA GAA ACT GTG AAG ATC IGCT TAT TAT GTC CTA TC-3′) that had been circularized as described (11). Twenty-five microliters was applied to each subarray and incubated for 30 min, and then microarrays were washed twice. The RCA reaction was carried out for 45 min. at 37° C. in a 25 μL volume containing T7 native DNA polyrnerase as described (12) in the presence of 0.05 uM detector probe 5′-Cy5-TGT CCT ATC CTC AGC TGG-Cy5. After washing, slides were scanned (General Scanning Luminomics, Watertown, Mass.) at a 10 μm resolution with a laser setting of 80 and a PMT setting of 60. Mean pixel fluorescence was quantified using the fixed circle method in QuantArray software (General Scanning).

Cell Culture. CD34⁺ stem cells were purified from leukapheresis products and frozen in aliquots of 2.5×10⁶ in PBS/20% human albumin/10% DMSO and stored in liquid nitrogen (14). Cells were thawed and cultured at 1×10⁴/ml/well in 24-well plates in X-VIVO-20 containing 100 ng/ml GM-CSF (granulocyte macrophage colony stimulating factor; 5.6 IU/mg), 20 ng/ml stem cell factor (5×10⁴ U/mg), 2.5 ng/ml TNF-α (2×10⁷ U/mg), 0.5 ng/ml TGF-β1 (2×10⁷ U/mg), and 100 ng/ml Flt3 ligand (“supplemented X-VIVO”). Cultures were incubated at 37° C. with 5% CO₂ in a humidified environment for 7-10 days. Clusters of immature Langerhans cells were purified (12) and then cultured at 5×10⁵ cells/ml/well in 24-well plates for an additional 2 days. For maturation, to supplemented X-VIVO was added an additional 12.5 ng/ml of TNF-α or 10 μg/ml of LPS (Sigma).

### Example 2

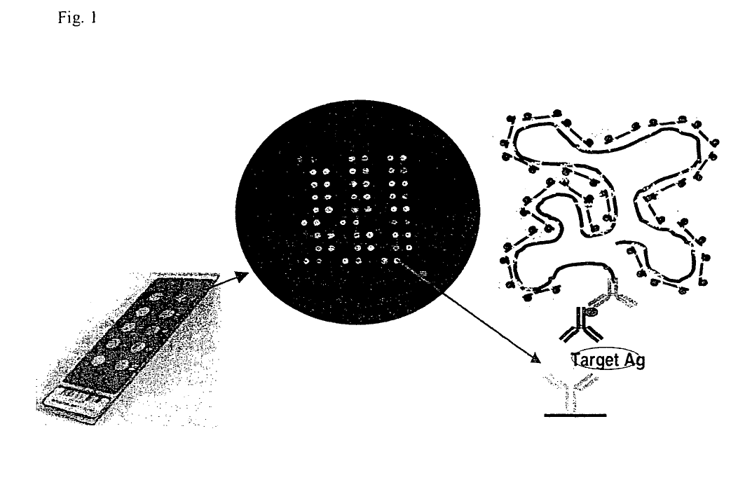

Antibody Microarrays. Microarrays were printed on thiolsilane-coated and cross-linker activated glass slides divided by Teflon boundaries into sixteen 0.5 cm diameter circular analysis sites (or “subarrays”; FIG. 1). This format minimized reagent consumption, segregated immunoassays into relatively small groups, and allowed different samples to be applied to each. Subarray spacing allowed automated processing by a liquid-handling robot with an 8-pipette tip head. Each microarray slide allowed duplicate measurements of 51 human cytokines in 8 samples. Cytokines representing both inflammatory and homeostatic groups were chosen for analysis, since they represented low abundance proteins whose absolute level was of biological significance (Table 1; 15-18). 150 “features” were printed at known locations in each subarray. 100 of these represented 25 single monoclonal antibodies, each spotted in quadruplicate and each specific for a single cytokine. Remaining features represented internal calibrators (dilutions of Cy5-labeled BSA (bovine serum albumin)) or controls for binding and signal amplification by the anti-biotin-DNA conjugate and RCA (biotin-labeled IgG). The latter features enabled standardization between subarrays and microarrays. Since features were printed in quadruplicate, each subarray allowed measurement of 25-26 cytokines. Coefficients of variation (CV), as measured by fluorescent intensity after incubation with Cy5-labeled goat anti-mouse antibody, were ˜10% for quadruplicate features on the array, while CVs between different antibodies were ˜25%. Antibody microarrays were stable for at least 1 month when stored at 4° C.

TABLE 1
Comparison of Protein Chip Results with Published Reports.

| Treatment | Study | Cytokine | Assay | Result | This Study |
|---|---|---|---|---|---|
| LPS | 14 | IL-12 | ELISA | 20 pg/ml at 48 hrs | 8 pg/ml at 72 hrs |
| | 25 | RANTES | ELISA | 400 ng/ml at 24 hrs | 300 pg/ml at 24 hrs |
| | | MIP-1β | ELISA | 5 μg/ml at 24 hrs | 300 pg/ml at 24 hrs |
| | | IP-10 | Northern | Up-regulated at 3 hrs | 500 pg/ml at 24 hrs |
| | | IL-8 | Northern | Up-regulated at 3 hrs | 5 ng/ml at 12 hrs |
| | | TARC | RT-PCR | Up-regulated at 30 hrs | 2 ng/ml at 24 hrs |
| | | MDC | RT-PCR | Up-regulated at 30 hrs | 20 ng/ml at 24 hrs |
| TNF-α | 25 | RANTES | ELISA | 10 ng/ml at 24 hrs | 80 pg/ml at 24 hrs |
| | | MIP-1β | ELISA | 1 μg/ml at 24 hrs | 50 pg/ml at 24 hrs |

Immunoassays for each of the 51 cytokines were performed with arrayed capture monoclonal antibodies and biotinylated, polyclonal, second antibodies. Since assays were performed simultaneously with a cocktail of 25 second antibodies applied to 25 capture antibodies on a subarray, antibodies were extensively evaluated for sensitivity, cross reactivity and non-specific signals using purified cytokines, and approximately one half were replaced. Most remaining non-specific signals were eliminated in the 2 groups of 25-26 immunoassays by iteratively switching between subarrays capture antibodies that gave non-specific signals with particular polyclonal second antibodies. Residual cross-reactivity and non-specific signals were minimized by optimization of washing and blocking conditions, antibody concentrations, and incubation times.

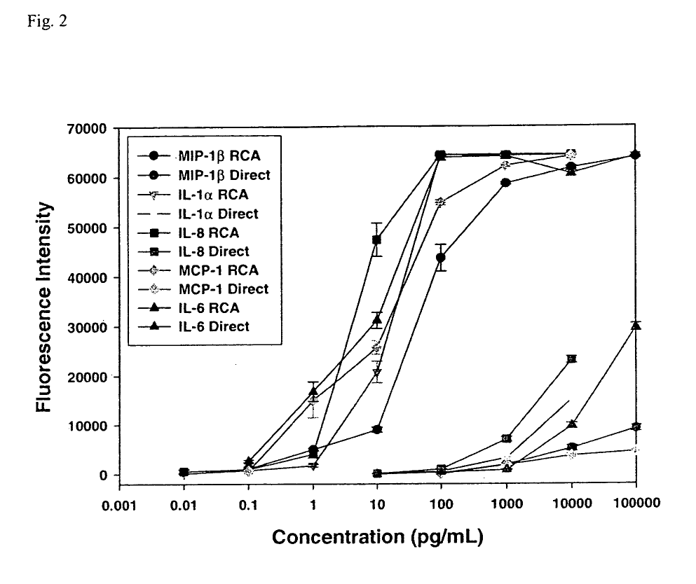

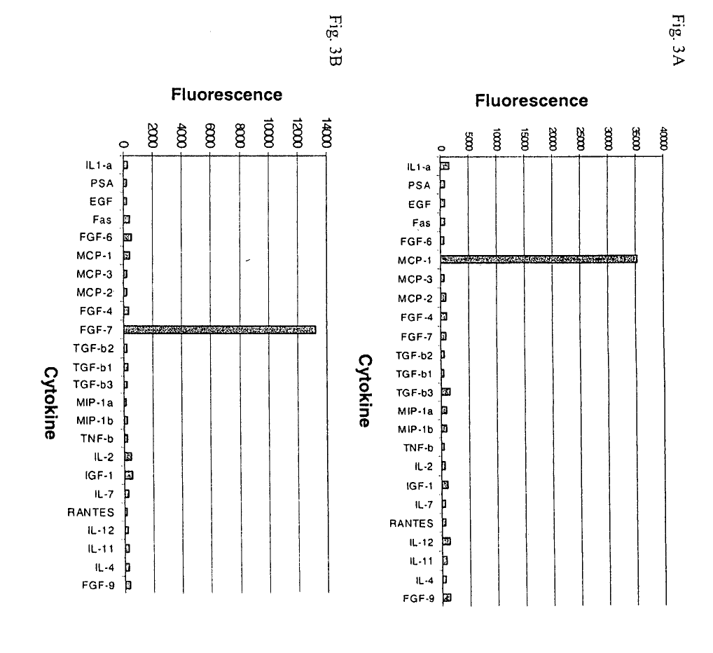

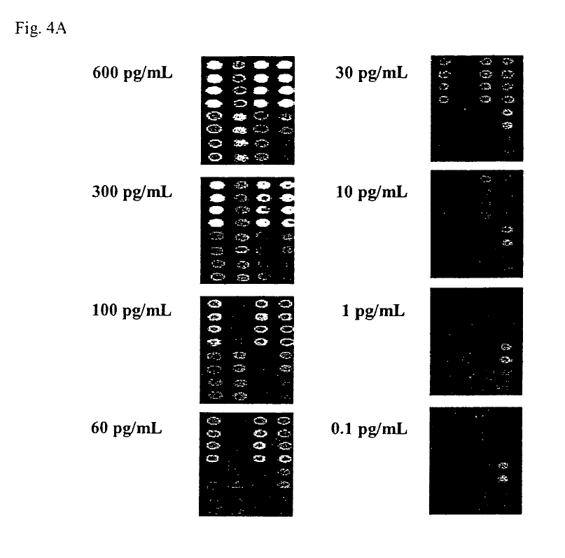

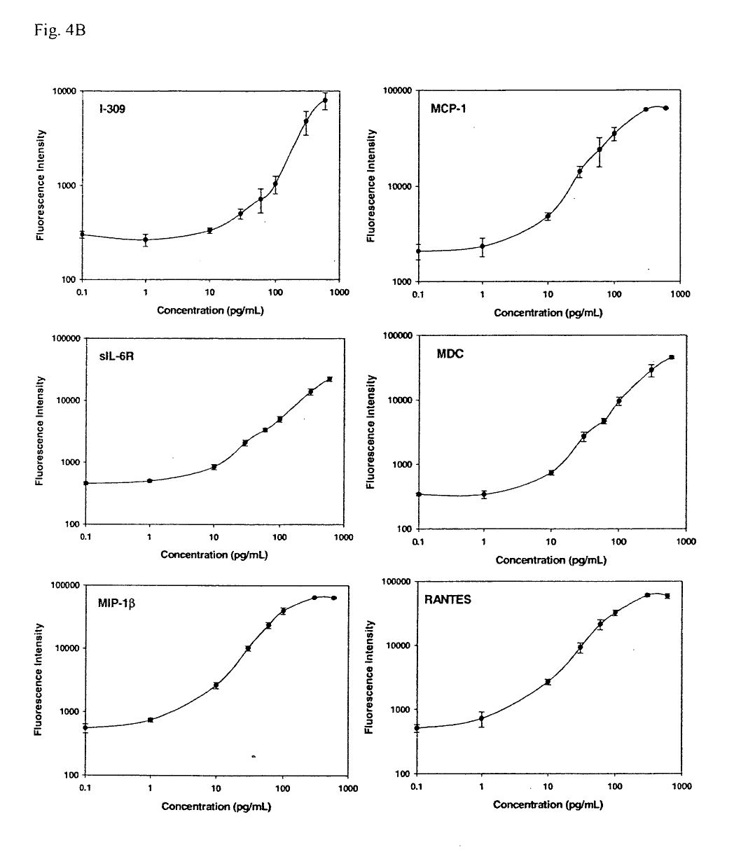

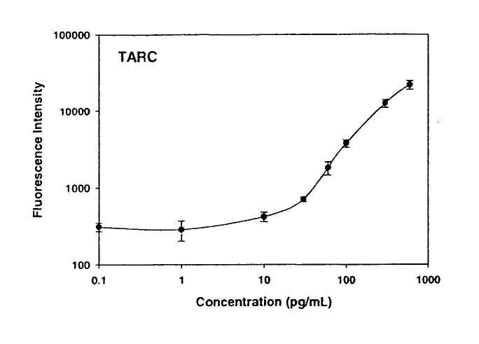

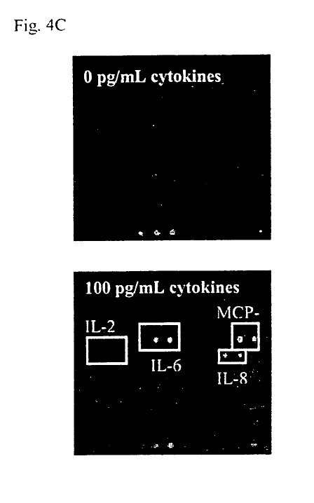

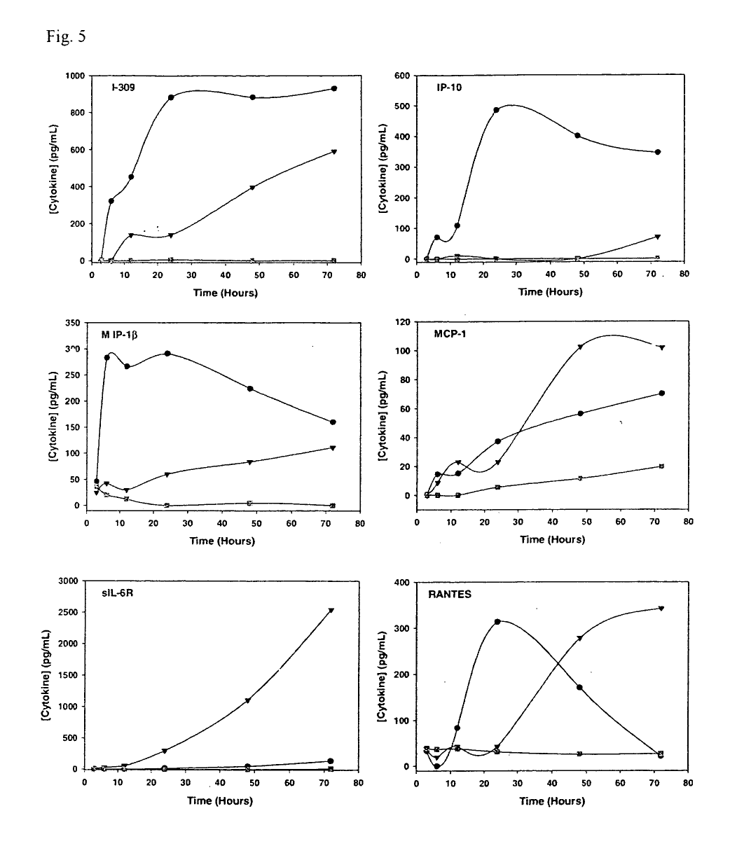

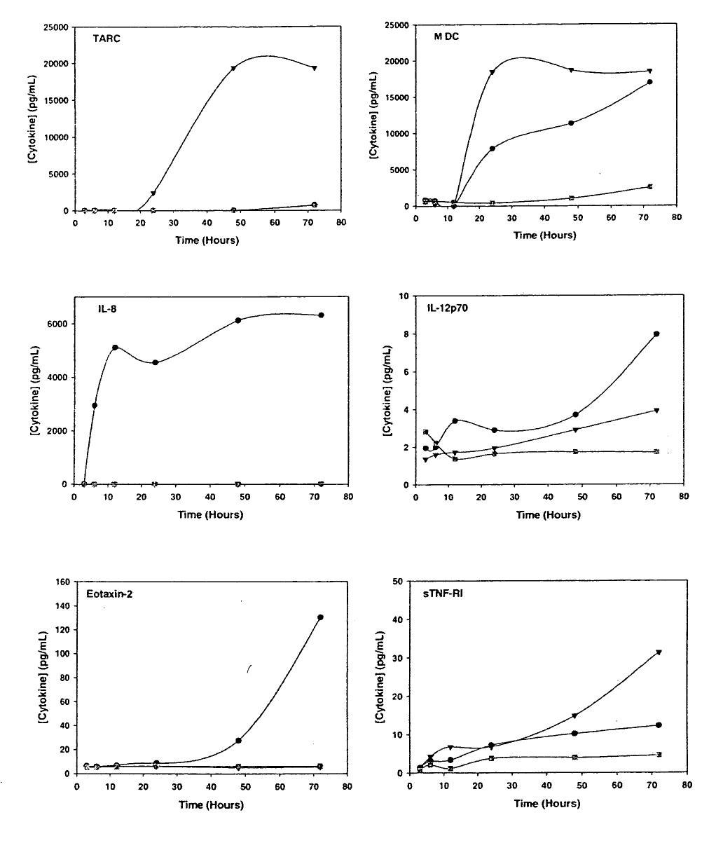

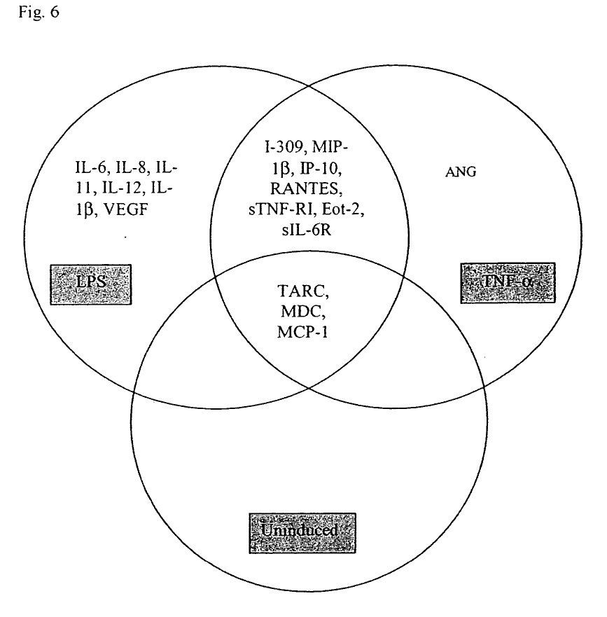

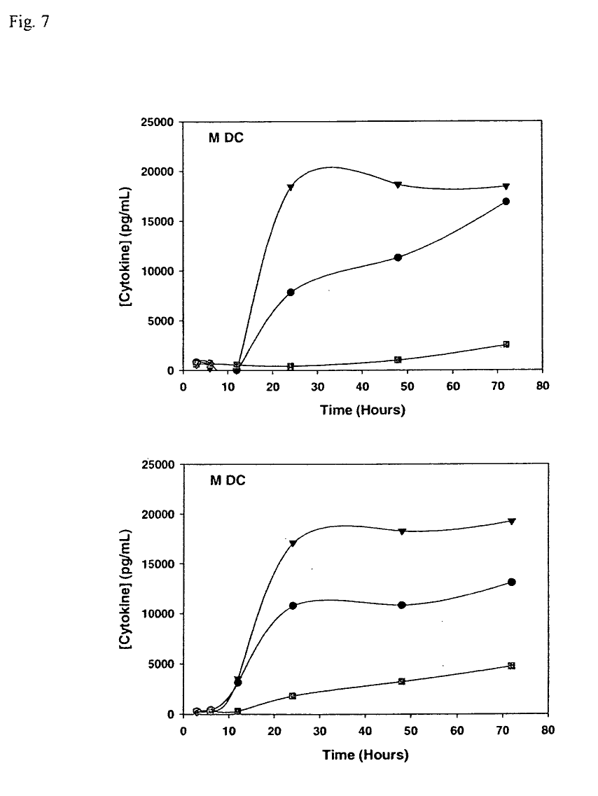

Sensitivity and specificity of cytokine detection by RCA. Amplification of immunoassay signals on microarrays was necessary since customary sensitivity without amplification was ˜1 ng/mL, which was insufficient for measurement of biologically-significant cytokine perturbations (15-18) (FIG. 2). Immunoassay sensitivity enhancement was achieved by covalent attachment of an oligonucleotide for RCA priming to an anti-biotin antibody with a stoichiometry of ˜5:1. Such attachment did not significantly alter the avidity of the anti-biotin antibody (12). RCA was performed with an 80 mer, synthetic DNA circle, and a DNA polymerase, yielding, as a single product, a long ribbon of single-stranded DNA for 45 minutes (11, 19). This product remained attached to the anti-biotin antibody and was detected by hybridization of complementary, fluorescent oligonucleotides to tandem copies of circle sequence (12). RCA and hybridization were performed at 37° C. in an isotonic buffer of neutral pH, and did not appreciably dissociate the immunoassay components (12). Non-specific signals related to RCA were significantly less than those related to antibody cross-reactivity, and were minimized by appropriate washing and blocking conditions (data not shown). RCA immunoassay sensitivity on microarrays, as measured by serial dilution of single, purified cytokines, was femtomolar, representing more than a 1000-fold improvement (FIG. 2A): 35 (70%) of the 51 cytokine features had a sensitivity of ≦10 pg/mL, 13 (25%) had a sensitivity of ≦100 pg/mL, and 3 (5%) had a sensitivity of ≦1000 pg/mL (Table 1). Importantly, the dynamic range and precision of RCA microarray immunoassays was similar to unamplified immunoassays (approximately 4 orders of magnitude dynamic range with an array scanner (FIG. 2) and precision of ˜5% CV for assays carried out on 4 arrays, even at low cytokine concentrations). Femtomolar sensitivity and 4 log dynamic range are adequate for measurement of most biologically relevant changes in cytokine secretion and, probably, for most biologically relevant changes in protein expression (Table 1; 20-22).

Specificity of RCA microarray immunoassays was examined in 3 ways: Firstly, microarrays were incubated with relatively high concentrations of 2 cytokines (1 ng/mL MCP-1 (macrophage chemoattractant protein) or FGF-7 (fibroblast growth factor-7)); following detection with 24 biotinylated antibodies and RCA, signals were only observed at the appropriate features, and signal to noise was >100:1 (FIG. 3B). Secondly, serial dilutions of a mixture of 7 cytokines were applied to microarrays; following detection with 7 biotinylated antibodies and RCA, dose-dependent signals were observed only at the appropriate features, and sensitivity for each was 1-10 pg/mL (FIG. 4A-B). Thirdly, a mixture of 10 cytokines (100 pg/mL) was applied to microarrays, but detection was with biotinylated polyclonal antibodies to only 4, followed by RCA. Signals were observed only where both cytokine and specific polyclonal antibody were present (FIG. 4C). Low, fixed, non-specific signal was detected with a few cytokines. This non-specific signal was eliminated by adsorbing secondary antibodies on mouse IgG (data not shown), but was unnecessary in practice since experimental designs sought changes in cytokine level.

### Example 3

Application of Antibody Arrays to Langerhans Cell Maturation. Dendritic cells are critical in both initiating and directing the immune response. Dendritic cells (DCs), which include multiple subsets (23), sample and process antigen and then, given the proper environmental cues, mature, migrate and present these antigens to naive T cell populations in draining lymph nodes. The cytokine microarray was used to study factors secreted during maturation of Langerhans cells (LC), a type of DC found in the epidermis of skin, to shed light on LC signals for helper T cell (T_{H}) maturation. The identification of such cytokines is significant because LCs elaborate numerous cytokines that affect both the development of LCs and lymphocytes in the surrounding area (23-24). Factors secreted by LC at certain stages of differentiation induce naive helper T lymphocytes (T_{H0}) to differentiate into either T_{H1} or T_{H2} cells that, in turn, stimulate cellular or humoral immune responses, respectively. Following antigen stimulation, the relative induction of T_{H1} and T_{H2} populations is believed to be an important determinant of immune response and immunopathology. Synchronized LC maturation can be induced in vitro by LPS or TNF-α (14); LPS induces LC differentiation to maturity, associated with cytokine secretion sufficient to stimulate T_{H0}→T_{H1} transition, whereas TNF-α treatment drives LCs to an intermediate stage of differentiation, associated with cytokine secretion that stimulates T_{H0}→T_{H2} transition (14, 24).

CD34⁺ cells from granulocyte colony stimulating factor- (G-CSF-) treated patients were cultured without fetal calf serum (to allow precise measurement of secreted proteins) in medium containing a defined cocktail of cytokines for 7-8 days. Resultant, immature LCs were isolated and recultured for 4 days in the presence of either LPS, TNF-α or unsupplemented growth medium, as previously described (14). Supematant samples were collected at 6 time points after start of reculture, and the levels of 51 cytokines in each sample were measured simultaneously in duplicate on the protein chips. The fluorescence intensity of microarray features was averaged for each feature and sample, and the resulting time courses of cytokine secretion were determined. For selected cytokines, fluorescence intensities were converted to protein levels via standard curves generated from serial dilutions of purified analytes in unsupplemented growth medium (FIG. 5).

Protein chip analysis revealed that about one-third of the cytokines represented on the array increased in abundance at least 4-fold during the 72-hour culture (FIG. 6). 16 cytokines were induced at least 4-fold by LPS, while 12 were induced by TNF-(α. Only 3 cytokines were secreted by LCs cultured without addition of LPS or TNF-(α; in no case was a cytokine secreted in untreated LCs but not in LPS or TNF-α treated cells. In contrast, 6 cytokines (IL-1β, IL-6, IL-8, IL-11, IL-12, and CTNF) were specifically induced by LPS, and 1 cytokine (ANC) was induced only by TNF-α (FIG. 6). Nine cytokines (Eot-2, I-309, IP-10 (interferon inducible protein-10), MCP-1. RANTES, MIP-1β (macrophage inducible protein 1β), IL-6sR, TARC (thymus and activation regulated chemokine), and MDC (macrophage derived chemokine)) were induced by both LPS and TNF-α, although the levels and time-course of secretion induced by the treatments were usually significantly different (FIG. 5). It was clear, therefore, that multiplexed measurements of cytokine secretion on these chips were useful for characterizing LC activity during induced differentiation.

### Example 4

Validation of the cytokine microarray. Because the protein chip platform used in this study was novel, it was important to validate the results obtained by comparison with the results of previous studies and conventional formats (Table 2). MDC, for example, was induced sufficiently to allow reliable confirmation of the microarray data with a less sensitive ELISA assay. Results from both assays were concordant (FIG. 7), although the ELISA assay would have consumed 1000-fold more sample to produce the data acquired with the microarray. MDC and a related chemokine TARC, have previously been shown by RT-PCR to be induced in DCs by LPS (Table 2; 25). Our results confirmed these findings, but also extended them by demonstrating that MDC and TARC are induced by TNF-α with faster kinetics and a greater abundance than LPS (FIG. 5). Concordance with previous studies was also found with IL-8, IP-10, IL-12, MIP-1β, and RANTES, all of which were induced with LPS (14, 25-26) (Table 2, FIG. 5). Of note were IL-12p70, RANTES and MIP-1β induction by LPS; previous studies have shown greater induction of these cytokines in monocyte-derived DCs than was observed with LCs in the current study. As has been shown for IL-12p70 (14), this may reflect differences between monocyte-derived DCs and LCs, rather than immunoassay platform differences. These discrepancies notwithstanding, the observed patterns of cytokine expression were consistent with previous studies of LPS- and TNF-α- treated LCs, attesting to the validity of the cytokine microarray method. Table 2 is shown below.

TABLE 1
Protein Microarray Content Information

| | | | ELISA | | Array | |
|---|---|---|---|---|---|---|
| CYTOKINE | ED₅₀* | | Sensitivity** | | Sensitivity | |
| ANG | Not available | | 6.0 | pg/mL. | 10 | pg/mL |
| AR | 5000-15000 | pg/mL. | Not avaIlable | | 50 | pg/mL |
| CNTF | 50000-150000 | pg/mL. | 8 | pg/mL. | 100 | pg/mL |
| EGF | 100-400 | pg/mL. | 0.7 | pg/mL. | 1 | pg/mL |
| ENA-78 | 3000-15000 | pg/mL. | 15 | pg/mL. | 10 | pg/mL |
| Eot | 10000-20000 | ng/mL. | 5 | pg/mL. | 0.5 | pg/mL |
| Eot-2 | 10000-50000 | pg/mL. | 8.5 | pg/mL. | 5 | pg/mL |
| FGF-6 | 100-300 | pg/mL. | Not available | | 100 | pg/mL |
| FGF-7 | 15000-25000 | pg/mL. | 15 | pg/mL. | 10 | pg/mL |
| FGF-9 | 1000-2000 | pg/mL. | Not available | | 100 | pg/mL |
| Flt-3 L | 500-1000 | pg/mL. | 7 | pg/mL. | 10 | pg/mL |
| G-CSF | 20-60 | pg/mL. | 20 | pg/mL. | 1000 | pg/mL |
| GDNF | 1000-3000 | pg/mL. | Not available | | 1 | pg/mL |
| GM-CSF | 20-80 | pg/mL. | 3 | pg/mL. | 3 | pg/mL |
| I-309 | 3000-9000 | pg/mL. | Not available | | 10 | pg/mL |
| IFN-gamma | 800-1500 | pg/mL. | 8 | pg/mL. | 10 | pg/mL |
| IL-10 | 500-1000 | pg/mL. | 3.9 | pg/mL. | 100 | pg/mL |
| IL-11 | 60-240 | pg/mL. | 8.0 | pg/mL. | 100 | pg/mL |
| IL-12 (p70) | 50-200 | pg/mL. | 5.0 | pg/mL. | 10 | pg/mL |
| IL-13 | 3000-6000 | pg/mL. | 32 | pg/mL. | 10 | pg/mL |
| IL-15 | 500-2000 | pg/mL. | 2 | pg/mL. | 60 | pg/mL |
| IL-16 | 2 × 10⁶-10⁶ | pg/mL. | 13.4 | pg/mL. | 500 | pg/mL |
| IL-17 | 2000-6000 | pg/mL. | 15 | pg/mL. | 10 | pg/mL |
| IL-18 | Not available | | 12.5 | pg/mL. | 100 | pg/mL |
| IL-1a | 3-7 | pg/mL. | 1.0 | pg/mL. | 1 | pg/mL |
| IL-1b | 5-10 | pg/mL. | 1 | pg/mL. | 10 | pg/mL |
| IL-1ra | 20000-60000 | pg/mL. | 22 | pg/mL. | 100 | pg/mL |
| IL-3 | 100-400 | pg/mL. | 7.4 | pg/mL. | 1000 | pg/mL |
| IL-4 | 50-200 | pg/mL. | 10 | pg/mL. | 10 | pg/mL |
| IL-6 | 200-800 | pg/mL. | 0.70 | pg/mL. | 1 | pg/mL |
| IL-6 sR | 5000-15000 | pg/mL. | 7 | pg/mL. | 1 | pg/mL |
| IL-7 | 200-500 | pg/mL. | 1.0 | pg/mL. | 10 | pg/mL |
| IL-8 | 100-500 | pg/mL. | 10 | pg/mL. | 0.5 | pg/mL |
| IP-10 | 3000-12000 | pg/mL. | 4.46 | pg/mL. | 6 | pg/mL |
| MCP-1 | 5000-20000 | pg/mL. | 5.0 | pg/mL. | 3 | pg/mL |
| MCP-2 | 30000-120000 | pg/mL. | Not available | | 10 | pg/mL |
| MCP-3 | 20000-80000 | pg/mL. | Not available | | 10 | pg/mL |
| M-CSF | 500-1500 | pg/mL. | 9 | pg/mL. | 10 | pg/mL |
| MDC | 3000-9000 | pg/mL. | 62.5 | pg/mL. | 5 | pg/mL |
| MIP-1a | 2000-10000 | pg/mL. | 10 | pg/mL. | 30 | pg/mL |
| MIP-1b | 10000-30000 | pg/mL. | 11.0 | pg/mL. | 1 | pg/mL |
| MIP-1d | 2000-8000 | pg/mL. | Not available | | 30 | pg/mL |
| OSM | 150-300 | pg/mL. | 6.0 | pg/mL. | 1 | pg/mL |
| RANTES | 10000-20000 | pg/mL. | 8 | pg/mL. | 3 | pg/mL |
| SCF | 2500-5000 | pg/mL. | 9.0 | pg/mL. | 10 | pg/mL |
| TARC | 3000-9000 | pg/mL. | 7 | pg/mL. | 10 | pg/mL |
| TGF-b1 | 5-20 | pg/mL. | 7 | pg/mL. | 10 | pg/mL |
| TNF-a | 20-50 | pg/mL. | 4.4 | pg/mL. | 10 | pg/mL |
| TNF-b | 20-50 | pg/mL. | 16 | pg/mL. | 30 | pg/mL |
| sTNF RI | 45000-90000 | pg/mL. | 3.0 | pg/mL. | 6 | pg/mL |
| VEGF | 5000-10000 | pg/mL. | 9.0 | pg/mL. | 30 | pg/mL |

*ED₅₀ information was obtained from product literature provided by R & D Systems.
**ELISA sensitivity information was obtained from product literature provided by R & D systems for the Quantikine Immunoassay Kits.

### Example 5

Biological relevance. The suggestion that quantitative expression proteomics surveys are of greater biological relevance than RNA level measurement (27), appeared to be supported in the current study by the ability not only to measure changes in protein abundance, but also to predict effects of these changes on biological activity: for example IP-10, whose RNA is known to be induced in LPS-stimulated DCs (25), was shown in the present study to be secreted at 0.5 ng/ml, which exceeds the ED₅₀ for induction of T_{H1} chemotaxis by IP-10 (18, 28-29). Another example was IL-8, which induces T-cell chemotaxis and suppresses T_{H0}→T_{H2} differentiation with an ED₅₀ of 0.1-0.5 ng/ml (30), and which was present in LPS-treated LC supernatants at a concentration of 6 ng/ml (FIG. 5). In contrast was MIP-1β, which, while induced significantly by both LPS and TNF-(α, achieved levels in supernatants (0.3 ng/ml) that were 10-fold below the ED₅₀ for chemotaxis of T_{H1} cells (28) (FIG. 5), which may indicate that MIP-1β secretion by LPS-induced LCs is irrelevant for T_{H1} chemotaxis. However, the ED₅₀ of cytokines measured in model assays in isolation should be interpreted with caution, since multiple cytokines may act synergistically.

Biologically relevant cytokine induction was also observed following TNF-β treatment. For example, MCP-1, which has not been previously reported to be secreted by DCs, was induced by TNF-(α at 0.1 ng/ml, a concentration sufficient to induce T_{H0}→T_{H2} differentiation (FIG. 5) (31-33). Similarly, induction of 20 ng/ml MDC and TARC by TNF-α should be sufficient to induce T_{H2} chemotaxis (FIG. 5; 34). Interestingly, RANTES, which was secreted by both LPS- and TNF-α-treated LCs in similar amounts (FIG. 5), is known to enhance both humoral- and cell-mediated immune responses (35-36).

RNA expression levels do not always correlate with protein levels (27, 37). In the present study, there were 2 examples of apparent discordance in abundance of mRNA and protein: IL-8 protein was rapidly induced by LPS and then remained at high levels throughout the culture period (FIG. 5). This contrasted with a previous study in which IL-8 mRNA levels of 1ps-treated monocyte-derived DCs peaked at 3 hrs after 1ps treatment, followed by a decline to baseline level at 30 hrs (25). The discordance between mRNA and protein levels at 30 hours may reflect ongoing secretion of stored intracellular IL-8, or IL-8 longevity in the culture media. Striking discordance was observed with RANTES, where mRNA levels had been found to gradually increase with time (25), whereas the protein level in culture supematants peaked 24 hours after LPS introduction and then declined. Under these conditions there appeared to be no correlation between RANTES transcription and translation/exocytosis. In general, measurement of protein, rather than RNA, abundance appeared to be of greater biological relevance.

An exciting application of the cytokine protein chip is in comparison of the abundance of a particular protein with that of its cognate receptor on putative target cells. For example, RANTES levels peaked 24 hours after LPS introduction and then declined. A previous report has shown that CCR1 and CCR5, receptors for RANTES, decrease in abundance on the surface of DCs within two hours of LPS exposure (38), suggesting the existence of an autocrine loop. Similarly, the RCA immunoassay was of sufficient sensitivity to enable the novel demonstration that 20 pg/mL eotaxin-2 was induced starting 48 hours after LPS introduction, and increasing to 140 pg/mL at 72 hours (FIG. 5). Of note is a recent report that after 48 hours of LPS stimulation, dendritic cells lose the capacity to promote T_{H0}→T_{H1} differentiation, and instead induce T_{H0}→T_{H2} differentiation (39). Eotaxin-2 provides a possible molecular explanation for this change since it has been shown to act specifically on T_{H2} lymphocytes expressing its receptor, CCR3 (18, 40). Finally, CCR4, the receptor for MDC and TARC, has been suggested to be a specific marker for T_{H2} lymphocytes (34, 41-43). Thus, the observation that TNF-α-induced LCs secrete MDC and TARC in biologically relevant amounts suggests a role for MDC and TARC in LC-induced T_{H0}→T_{H2} differentiation or recruitment.

Other novel and intriguing cytokine-cytokine receptor findings were that two soluble receptors, sIL-6R (soluble interleukin-6 receptor) and sTNF-RI (soluble tumor necrosis factor receptor-1), were specifically induced by TNF-α (tumor necrosis factor-α; FIG. 5). Induction of soluble IL-6R, but not IL-6, by TNF-α is of interest since IL-6 was induced by LPS, and sIL-6R is known to cause maturing DCs to differentiate to macrophages (44). Specific induction of sTNF-RI (soluble tumor necrosis factor receptor-1) by TNF-α was not an unexpected finding considering that the activity of TNF-(α is known to be modulated by proteolytic shedding of the soluble extracellular domain of its receptor (45). Finally, 1-309 was secreted at relatively high levels by both LPS- and TNF-α-treated LCs (FIG. 5). This chemokine was recently implicated in recruiting a CCR8⁺ T_{H} subtype characterized by the production of high levels of IL-10 and low amounts of IFN-γ and IL-4 (46). Thus, 1-309 also appears to be a novel product of stimulated LCs of potential biological importance.

In summary, the application of an RCA cytokine chip to the study of LC maturation illustrated several advantages over conventional assays: Firstly, universal RCA signal amplification was shown to be compatible with protein arrays. Secondly, RCA-amplified protein arrays allowed, for the first time, 51 members of a family of proteins to be measured simultaneously without compromise of biologically-relevant sensitivity. Because cytokines in cocktails can elicit biological effects that are different from those observed in isolation, global patterns of cytokine expression are more likely to yield biologically relevant and clinically useful information than assays of single cytokines. Of note, we have recently increased the number of cytokines measured on protein chips to 75 with similar performance (data not shown). Thirdly, multiplexed protein measurement was shown to elicit biological information beyond that obtained with the most prevalent method for surveying gene activity, the measurement of RNA levels. The cytokine chip should be useful in a variety of studies of basic immunology, infection, autoimmunity, immunodeficiency, and inflammation. Additional chips, featuring 50-100 RCA sandwich immunoassays, are planned that will focus on other collections of proteins that mediate signal transduction, apoptosis, and toxic drug responses. Finally, chip-based RCA signal amplification may also prove useful for chips that examine protein-protein or protein-drug interactions.

While the invention has been described with respect to specific examples including presently preferred modes of carrying out the invention, those skilled in the art will appreciate that there are numerous variations and permutations of the above described systems and techniques that fall within the spirit and scope of the invention as set forth in the appended claims.

### REFERENCES

1. Bussow, K., Cahill, D., Nietfeld, W., Bancroft, D., Scherzinger, E., Lehrach, H. & Walter, G. A method for global protein expression and antibody screening on high-density filters of an arrayed cDNA library. Nucleic Acids Res. 26, 5007-5008 (1998).

2. Leuking, A., Hom, M., Eickhoff, H., Buessow, K., Leharch, H. & Walter, G. Protein microarrays for gene expression and antibody screening. Anal. Biochem. 270, 103-111 (1999).

3. Ge, H. UPA, a universal protein array system for quantitative detection of protein-protein, protein-DNA, protein-RNA and protein-ligand interactions. Nucleic Acids Res. 28, (2000).

4. de Wildt, R. M. T., Mundy, C. R., Gorick, B. D., & Tomlinson, I. M. Antibody arrays for high-throughput screening of antibody-antigen interactions. Nat. Biotech. 18, 989-994 (2000).

5. MacBeath, G. & Schreiber, S. L. Printing proteins as microarrays for high-throughput function determination. Science 289, 1760-1763 (2000).

6. Zhu, H. et al. Global Analysis of Protein Activities Using Proteome Chips. Science 293, 2101-2105 (2001).

7. Silzel, J. W., Cercek, B., Dodson, C., Tsay, T., & Obremski, R. J. Mass-sensing, multianalyte microarray immunoassay with imaging detection. Clin. Chem. 44, 2036-2043 (1998).

8. Mendoza, L. G., McQuary, P., Mongan, A., R. Gangadharan, R., Brignac, S. & Eggers, M. High-throughput microarray-based enzyme-linked immunoabsorbant assay (ELISA). Biotechniques 27, 778-788 (1999).

9. Haab, B. B., Dunham, M. J. & Brown P. O. Protein microarrays for highly parallel detection and quantitation of specific proteins and antibodies in complex solutions. Genome Biology 2, 1-13 (2001).

10. Wiese, R., Belosludtsev, Y., Powdrill, T., Thompson, P. & Hogan, M. Simultaneous multianalyte ELISA performed on a microarray platform. Clin. Chem. 47, 1451-1457 (2001).

11. Lizardi, P., Huang, X., Zhu, Z., Bray-Ward, P., Thomas, D., & Ward, D. Mutation detection and single molecule counting using isothermal rolling circle amplification. Nature Genet. 19, 225-232 (1998)

12. Schweitzer, B., Wiltshire, S., Lambert, J., O'Malley, S., Kukanskis, K., Zhu, Z., Kingsmore, S. F., Lizardi, P. M. & Ward, D. C. Immunoassays with rolling circle DNA amplification: a versatile platform for ultrasensitive antigen detection. Proc. Natl. Acad. Sci. (USA) 97, 10113-10119 (2000).

13. Wiltshire, S., O'Malley, S., Lambert, J., Kukanskis, K., Edgar, D., Kingsmore, S. & Schweitzer, B. Detection of multiple allergen-specific IgE on microarrays by immunoassay with rolling circle amplification. Clin. Chem. 46, 1990-1993 (2000).

14. Gatti, E., Velleca, M. A., Biedermann, B. C., Ma, W., Unternaehrer, J., Ebersold, M. W., Medzhitov, R., Pober, J. S. & Mellman, I. Large-scale culture and selective maturation of human Langerhans cells from granulocyte colony-stimulating factor-mobilized CD34⁺ progenitors. J. Immunol. 164, 3600-3607 (2000).

15. Foster, J. R. The functions of cytokines and their uses in toxicology. Int. J. Exp. Pathol. 82, 171-192 (2001).

16. Pulendran, B., Palucka, K. & Banchereau, J. Sensing pathogens and tuning immune responses. Science 293, 253-256 (2001).

17. Zlotnik, A. & Yoshie, 0. Chemokines: A new classification system and their role in immunity. Immunity. 12, 121-127 (2000).

18. Moser, B. & Loetscher, P. Lymphocyte traffic control by chemokines. Nature Immunol. 2, 123-127 (2001).

19. Dean, F. B., Nelson, J. R., Giesler, T. L. & Lasken, R. S. Rapid amplification of plasmid and phage DNA using phi29 DNA polymerase and multiply-primed rolling circle amplification. Genome Res. 11, 1095-1099 (2001).

20. Naaby-Hansen, S., Waterfield, M. D. & Cramer, R. Proteomics—post-genomic cartography to understand gene function. Trends Pharmacol. Sci. 22, 376-384 (2001).

21. Figeys, D. Array and lab on a chip technology for protein characterization. Curr. Opin. Mol. Ther. 1, 685-694 (1999).

22. Emili, A. Q. & Cagney G. Large scale functional analysis using peptide or protein arrays. Nat. Biotechno. 18, 393-397 (2000).

23. Liu, Y-J. Dendritic cell subsets and lineages, and their functions in innate and adaptive immunity. Cell 106, 259-262 (2001).

24. Mellman, I., and Steinman, R. M. Dendritic cells: Specialized and regulated antigen processing machines. Cell 106, 255-258 (2001).

25. Sallusto, F. et al. Distinct patterns and kinetics of chemokine production regulate dendritic cell function. Eur. J. Immunol. 29, 1617-1625 (1999).

26. Moser, M. & Murphy, K. M. Dendritic cell regulation of T_{H1}-T_{H2} development. Nat Immunol. 1, 199-205 (2000).

27. Gygi, S. P., Rochon, Y., Franza, B. R. &. Aebersold R. Correlation between protein and mRNA abundance in yeast. Mol. Cell. Biol. 19, 1720-1730 (1999).

28. Sebastiani, S. et al. Chemokine receptor expression and function in CD4⁺ T lymphocytes with regulatory activity. J. Immunol. 166, 996-1002 (2001).

29. Loetscher, P., Pellegrino, A., Gong, J. H, Mattioli, I., Loetscher, M., Bardi, G., Baggiolini, M. & Clark-Lewis, I. The ligands of CXC chemokine receptor 3, I-TAC, Mig, and IP10, are natural antagonists for CCR3. J Biol Chem. 276, 2986-2991 (2001)

30. Gesser B. et al. IL-8 induces T cell chemotaxis, suppresses IL-4, and up-regulates IL-8 production by CD4⁺ T cells. J. Leukoc. Biol. 59, 407-411 (1996).

31. Matsushima, K., Larsen, C. G., DuBois, G. C. & Oppenheim, J. J. Purification and characterization of a novel monocyte chemotactic and activating factor produced by a human myelomonocytic cell line. J. Exp. Med. 169, 1485-1490 (1989).

32. Karpus, W. J. & Kennedy, K. J. MIP-1 alpha and MCP-1 differentially regulate acute and relapsing autoimmune encephalomyelitis as well as Th1/Th2 lymphocyte differentiation. J. Leukoc. Biol. 62, 681-687 (1997).

33. Gu, L., Tseng, S., Horner, R. M., Tam, C., Loda, M. & Rollins, B. J. Control of T_{H2} polarization by the chemokine monocyte chemoattractant protein-1. Nature 404, 407-411 (2000).

34. Imai, T., Nagira, M., Takagi, S., Kakizaki, Nishimura, M., Wang, J. Gray, P. W., Matsushima, K., & Yoshie, O. Selective recruitment of CCR4-bearing Th2 cells toward antigen-presenting cells by the CC chemokines thymus and activation-regulated chemokine and macrophage-derived chemokine. Int. Immunol. 11, 81-88, (1999).

35. Lillard, J. W. Jr., Boyaka, P. N., Taub, D. D. & McGhee, J. R. RANTES potentiates antigen-specific mucosal immune responses. J. Immunol. 166, 162-169 (2001).

36. Kawai, T., Seki, M., Hiromatsu, K., Eastcott, J. W., Watts, G. F., Sugai, M., Smith, D. J., Porcelli, S. A. & Taubman, M. A. Selective diapedesis of Th1 cells induced by endotbelial cell RANTES. J. Immunol. 163, 3269-3278 (1999).

37. Harford, J. B. & Morris, D. R. Post-transcriptional gene regulation. Wiley-Liss, Inc., New York, N.Y. (1997).

38. Sallusto, F., & Lanzavecchia, A. Understanding dendritic cell and T-lymphocyte traffic through the analysis of chemokine receptor expression. Immunol. Rev. 177, 134-140 (2000).

39. Langenkamp, A., Messi, M., Lanzavecchia, A. & Sallusto, F. Kinetics of dendritic cell activation: impact on priming of T_{H1}, T_{H2} and nonpolarized T cells. Nat Immunol. 1, 311-316 (2000).

40. Sallusto, F., Mackay, C. R., & Lanzavecchia, A. Selective expression of the cotaxin receptor CCR3 by human T helper 2 cells. Science 277, 2005-2008 (1997).

41. Andrew, D. P., Chang, M. S., McNinch, J., Wathen, S. T., Rihanek, M., Tseng, J., Spellberg, J. P. & Elias, C. G. STCP-1 (MDC) CC chemokine acts specifically on chronically activated Th2 lymphocytes and is produced by monocytes on stimulation with Th2 cytokines IL-4 and IL-13. J. Immunol. 161, 5027-5038 (1998).

42. Imai, T., Chantry, D., Raport, C. J., Wood, C. L., Nishimura, M., Godiska, R., Yoshie, O., & Gray, P. W. Macrophage-derived chemokine is a functional ligand for the CC chemokine receptor 4. J. Biol. Chem. 273, 1764-1768 (1998).

43. Baba, M., Imai, T., Nishimura, M., Kakizaki, M., Takagi, S., Hieshima, K., Nomiyama, H., & Yoshie, O. J. Biol. Chem. 272, 14893-14898 (1997).

44. Chomarat, P. et al. IL-6 switches the differentiation of monocytes from dendritic cells to macrophages. Nat. Immunol. 1, 510-514 (2000).

45. Thijs, L. G. & Hack, C. E. Time course of cytokine levels in sepsis. Intensive Care Med. Suppl 2, S258-63 (1995).

46. Cavani, A. et al. Patients with allergic contact dermatitis to nickel and non allergic individuals display different nickel-specific T cell responses: evidence for the presenceof effector CD8⁺ and regulatory CD4⁺ T cells. J. Invest. Dermatol. 111, 621-628 (2000).

1. A method of diagnosing an inflammatory syndrome in a patient, comprising:
determining in a test sample from a patient altered expression of one or more cytokines selected from the group consisting of: IL-6, IL-11, IL-1β, VEGF, ANG, I-309, sTNF-R1, Eot-2, and sIL-6R; and
using determination of said altered expression to diagnose an inflammatory syndrome in the patient.

2. The method of claim 1 wherein the syndrome is sepsis.

3. The method of claim 1 wherein the syndrome is arthritis.

4. The method of claim 1 wherein the syndrome is allergy.

5. The method of claim 1 wherein the syndrome is enteritis.

6. The method of claim 1 wherein the syndrome is severe acute pancreatitis.

7. The method of claim 1 wherein the syndrome is emphysema.

8. The method of claim 1 wherein the syndrome is multiple organ failure.

9. The method of claim 1 wherein the syndrome is acute respiratory distress syndrome (ARDS).

10. The method of claim 1 wherein the test sample is selected from the group consisting of serum, plasma, lymph fluid, peripheral lymphatic tissue, and blood.

11. The method of claim 1 wherein said altered expression is an increase in the level of said cytokine relative to other cytokines in the test sample.

12. The method of claim 1 wherein said altered expression is a decrease in the level of said cytokine relative to other cytokines in the test sample.

13. The method of claim 1 wherein said altered expression is determined relative to a test sample from a healthy individual.

14. The method of claim 1 wherein said altered expression is determined relative to a test sample from at least two healthy individuals.

15. The method of claim 1 wherein the test sample comprises dendritic cells.

16. The method of claim 1 wherein the test sample comprises Langerhans cells.

17. The method of claim 1 wherein altered expression is determined relative to a control sample from an organ or subject not experiencing an inflammatory syndrome.

18. The method of claim 17 wherein altered expression is determined if at least two-fold more cytokine is present in the test sample than in the control sample.

19. The method of claim 17 wherein altered expression is determined if at least three-fold more cytokine is present in the test sample than in the control sample.

20. The method of claim 17 wherein altered expression is determined if at least four-fold more cytokine is present in the test sample than in the control sample.

21. The method of claim 1 wherein said step of determining involves measuring amount of said cytokine in the test sample.

22. The method of claim 21 wherein the amount of said cytokine in the test sample is compared to amount of said cytokine in a control sample.

23. The method of claim 21 wherein the amount of said cytokine in the test sample is compared to amount of a control cytokine in the test sample.

24. The method of claim 1 wherein said step of determining employs an array of a first set of antibodies for capturing said cytokines.

25. The method of claim 24 wherein said step of determining employs a second set of antibodies which is applied to the array after binding of cytokines in the test sample to the first set of antibodies.

26. The method of claim 25 wherein said second set of antibodies comprises covalently attached oligonucleotides.

27. The method of claim 25 wherein a third set of antibodies is applied to the array which specifically bind to the second set of antibodies.

28. The method of claim 27 wherein the third set of antibodies comprises covalently attached oligonucleotides.

29. The method of claim 26 wherein rolling circle amplification is performed using said oligonucleotide as a primer.

30. The method of claim 28 wherein rolling circle amplification is performed using said oligonucleotide as a primer.

31. A method of treating a patient with an inflammatory syndrome, comprising:
administering to a patient with an inflammatory syndrome one or more antibodies which specifically bind to a cytokine selected from the group consisting of: IL-6, IL-11, IL-1β, VEGF, ANG, 1-309, sTNF-R1, Eot-2, and sIL-6R, whereby amount of said cytokine in said patient is reduced.

32. The method of claim 31 wherein the inflammatory syndrome is sepsis.

33. The method of claim 31 wherein the inflammatory syndrome is arthritis.

34. The method of claim 31 wherein the inflammatory syndrome is allergy.

35. The method of claim 31 wherein the inflammatory syndrome is enteritis.

36. The method of claim 31 wherein the inflammatory syndrome is severe acute pancreatitis.

37. The method of claim 31 wherein the inflammatory syndrome is emphysema.

38. The method of claim 31 wherein the inflammatory syndrome is multiple organ failure.

39. The method of claim 31 wherein the inflammatory syndrome is acute respiratory distress syndrome (ARDS).

40. The method of claim 31 wherein the antibodies are monoclonal antibodies.

41. The method of claim 31 wherein the antibodies are a cocktail of monoclonal antibodies.


SEQUENCE LISTING (3 sequences)

SEQ ID NO: 1 - DNA, 35 bp, Artificial Sequence
  rolling circle amplification reagents
aaaaaaaaaaaaaaacacagctgaggataggacat

SEQ ID NO: 2 - DNA, 80 bp, Artificial Sequence
  rolling circle amplification reagents
ctcagctgtgtaacaacatgaagattgtaggtcagaactcacctgttagaaactgtgaagatcgcttattatgtcctatc

SEQ ID NO: 3 - DNA, 18 bp, Artificial Sequence
  probe
tgtcctatcctcagctgg